(19) United States
(12) Patent Application Publication
(10) Pub. No.: US 2002/0143327 A1
(43) Pub. Date: Oct. 3, 2002

(54) Transverse connector for use in spinal corrective surgery

(75) Inventor: Alan E. Shluzas, Millis, MA (US)

Correspondence Address: TAROLLI, SUNDHEIM, COVELL, TUMMINO & SZABO L.L.P., 1111 LEADER BLDG.,, 526 SUPERIOR AVENUE, CLEVELAND, OH, 44114-1400, US

(73) Assignee: Endius Incorporated

(21) Appl. No.: 09/818,877

(22) Filed: Mar. 27, 2001

(51) Int. Cl. A61B 17/70

(52) U.S. Cl. 606/61

(57) ABSTRACT

An apparatus (10) for use in spinal corrective surgery includes two spinal rods (14, 16) at least partially extending along a vertebral column (18). A plurality of fixation elements fix the spinal rods (14, 16) to the vertebral column (18). A transverse connector (12) connects the spinal rods (14, 16) to each other. The transverse connector (12) has a first clamping member (24) with a spinal rod receiving channel for receiving one of the spinal rods (14). A connecting rod (28) integral with the first clamping member (24) extends transverse to the spinal rods (14, 16). The connecting rod (24) has a tapered portion (60) extending from the first clamping member (24). A first fastener (44) connects one of the spinal rods (14) to the first clamping member (24). A second clamping member (26) includes a spinal rod receiving channel (80) for receiving the other one of the spinal rods (16). The second clamping member (26) includes an opening (65) for receiving the connecting rod (28). A second fastener (82) connects the other one of the spinal rods (16) and the connecting rod (28) to the second clamping member (26).

### TECHNICAL FIELD

The present invention relates to an apparatus for use in spinal corrective surgery. In particular, the present invention relates to a transverse connector which is used to connect two spinal rods extending along a vertebral column.

### BACKGROUND OF THE INVENTION

Spinal corrective devices typically include two spinal rods fixed to a vertebral column by screws or hooks. The spinal rods are interconnected by transverse connectors. The device is typically implanted in a generally rectangular arrangement. This arrangement must have sufficient mechanical strength to support the vertebral column.

### SUMMARY OF THE INVENTION

The present invention is an apparatus for use in spinal corrective surgery. Two spinal rods at least partially extend along a vertebral column. A plurality of fixation elements fix the spinal rods to the vertebral column. A transverse connector connects the spinal rods to each other.

The transverse connector has a first clamping member with a spinal rod receiving channel for receiving one of the spinal rods. A connecting rod integral with the first clamping member extends transverse to the spinal rods. The connecting rod has a tapered portion extending from the first clamping member. A first fastener connects one of the spinal rods to the first clamping member. A second clamping member includes a spinal rod receiving channel for receiving the other one of the spinal rods. The second clamping member includes an opening for receiving the connecting rod. A second fastener connects the other one of the spinal rods and the connecting rod to the second clamping member.

### BRIEF DESCRIPTION OF THE DRAWINGS

The foregoing and other features of the present invention will become apparent to those skilled in the art to which the present invention relates upon reading the following description of the invention with reference to the accompanying drawings, in which:

FIG. 1 is a schematic illustration of an apparatus constructed in accordance with the present invention including a transverse connector attached to a vertebral column;

FIG. 2 is a schematic perspective view of the transverse connector of FIG. 1;

FIG. 3 is a schematic plan view of the transverse connector of FIG. 1;

FIG. 4 is a schematic view of the transverse connector taken along the lines 4-4 in FIG. 3;

FIG. 5 is a schematic view of the transverse connector taken along the lines 5-5 of FIG. 4.

### DESCRIPTION OF THE INVENTION

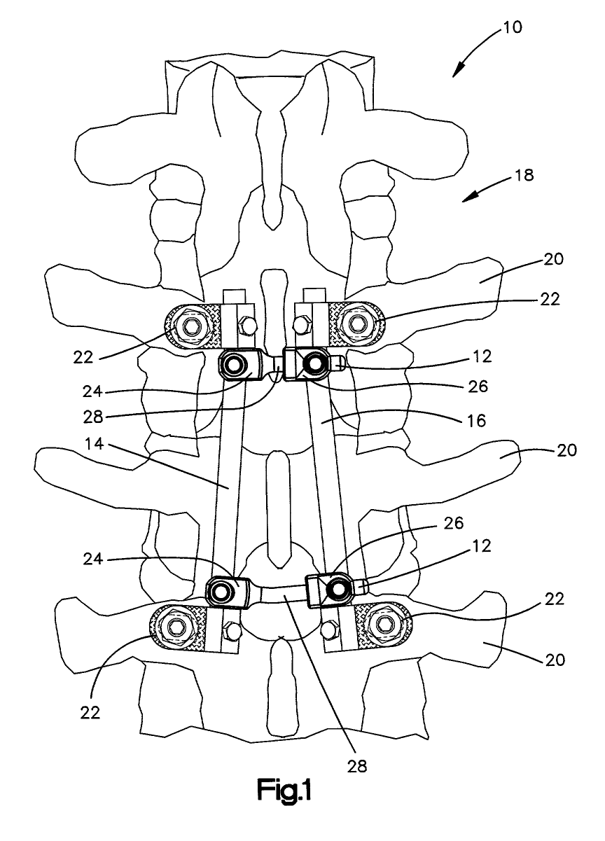

The present invention relates to an apparatus 10 for use in spinal corrective surgery. In particular, the present invention relates to a transverse connector 12 which is used to connect two spinal rods 14, 16 extending along a vertebral column 18. As representative of the present invention, FIG. 1 illustrates three vertebrae 20 secured together by the apparatus 10.

The spinal rods 14, 16 are fixed to the vertebrae 20 by fixation elements 22. The spinal rods 14, 16 are circular rods of a length sufficient to span a length of the vertebral column 18 to be stabilized. The spinal rods 14, 16 could be made of any suitable biocompatible material.

The fixation elements 22 can be of a type generally known in the art. For example, the fixation elements 22 can be threaded screw assemblies which are screwed into the desired vertebrae 20. Each screw assembly includes a threaded fastener or pedicle screw placed in a vertebra. Each screw assembly further includes a clamp for connecting the pedicle screw to a spinal rod and a nut for securing the pedicle screw to the clamp. The nut threadably engages the pedicle screw.

The apparatus 10 must have sufficient mechanical strength to support the vertebral column 18. For additional stabilization to the spinal rods 14, 16 against torsional rotation, transverse connectors 12 are connected to the spinal rods. The transverse connectors 12 are connected to the spinal rods 14, 16 to form a rectangular arrangement (FIG. 1).

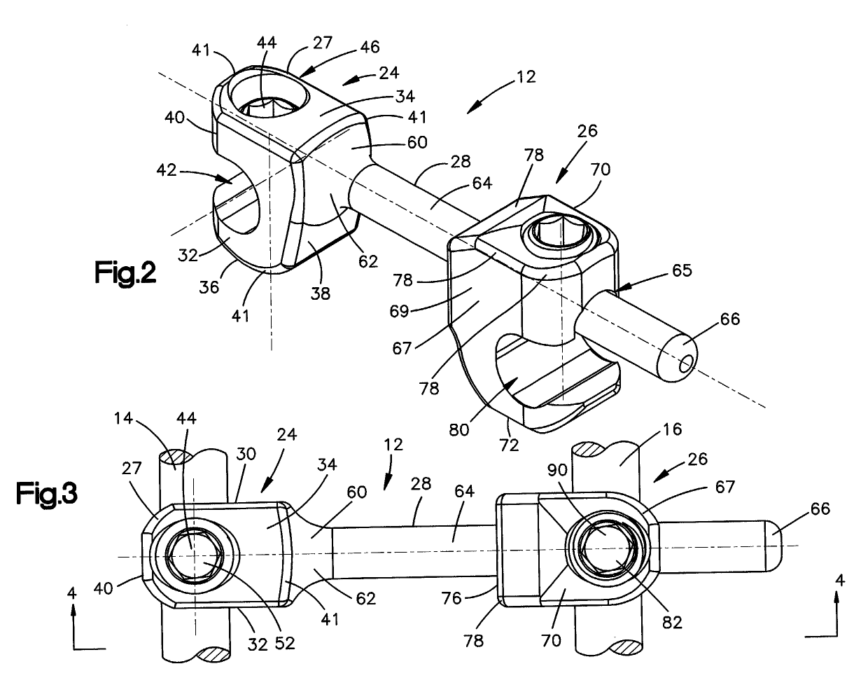

Each transverse connector 12 (FIGS. 2-4) includes first and second clamping members 24, 26. Each of the first and second clamping members 24, 26 has a C-hook structure. The first clamping member 24 is secured to the spinal rod 14. The second clamping member 26 is secured to the spinal rod 16. A connecting rod 28, integral with the first clamping member 24, extends transverse to the spinal rods 14, 16 between the first and second clamping members 24, 26.

The first clamping member 24 has an outer surface 27 (FIGS. 2-4). The outer surface 27 comprises a top portion 30, a base portion 32, a posterior portion 34 (FIG. 3), a frontal portion 36 and first and second lateral portions 38, 40. The portions 30, 32, 34, 36, 38, and 40 meet at edges 41. The edges 41 are beveled. Accordingly, the first clamping member 24 does not have sharp edges.

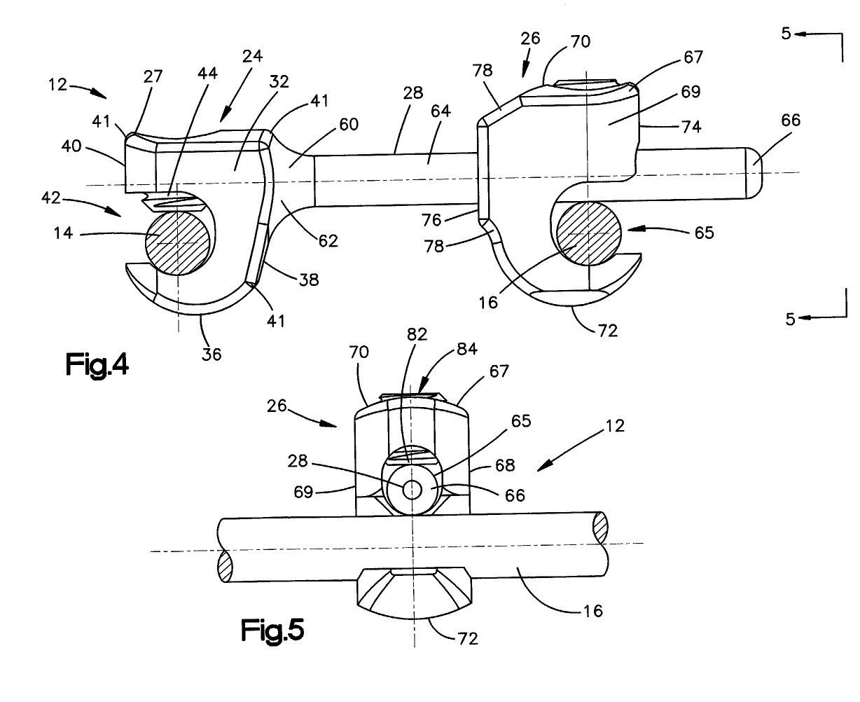

The first clamping member 24 (FIGS. 2 and 4) includes a spinal rod receiving channel 42 which receives the spinal rod 14. The first clamping member 24 is fastened to the spinal rod 14 by a first fastener or set screw 44 received in a bore 46. The bore 46 extends through the posterior portion 34 of the outer surface 27 of the first clamping member 24.

The set screw 44 (FIGS. 3 and 4) has external threads which engage internal threads in the bore 46 of the first clamping member 24. The head 52 of the set screw 44 can accommodate a hex driver or an Allen wrench. As the set screw 44 is tightened relative to the first clamping member 24, the set screw applies force to press the spinal rod 14 into position against the first clamping member.

The overall height of the first clamping member 24 is minimal since the connecting rod 28 is integral with the first clamping member, The bore 46 for receiving the set screw 44 sits low in the first clamping member 24 so the set screw can be driven low into the spinal rod receiving channel 42 (FIG. 4). Accordingly, the first clamping member 24 is arranged as close to the vertebral column 18 as possible.

The spinal rod receiving channel 42 of the first clamping member 24 has a height which is at least as large as the diameter of the spinal rod 14. The spinal rod receiving channel 42 has a contour which matches the contour of the spinal rod 14. When tightened, the set screw 44 protrudes slightly into the spinal rod receiving channel 42 (FIG. 4).

The connecting rod 28 is integrally formed with the first clamping member 24 (FIGS. 2-4). Accordingly, the connecting rod 28 and the first clamping member 24 are in one-piece. The connecting rod 28 extends from the first lateral portion 38 of the outer surface 27 of the first clamping member 24 opposite the spinal rod receiving channel 42 (FIG. 4).

The connecting rod 28 (FIGS. 2-4) has a tapered portion 60 extending from the first clamping member 24. The tapered portion 60 has a first cross-section 62 extending from the first lateral portion 38 of the outer surface 27 of the first clamping member 24. The connecting rod 28 has a second cross-section 64 spaced from the first clamping member 24 smaller than the first cross-section 62. The connecting rod 28 has a rounded end 66 opposite the tapered portion 60. Accordingly, the connecting rod 28 does not have any sharp edges.

The connecting rod 28 is a cylindrical rod. However, the connecting rod 28 can have various shapes and may be for example, a square-shaped rod. The connecting rod 28 is deformable to allow a slight bending of the connecting rod 28 if desired. The connecting rod 28 has a length to accommodate different spans between the spinal rods 14, 16.

The second clamping member 26 includes an opening 65 for receiving the connecting rod 28. The connecting rod 28 is received in the opening 65 so that the connecting rod 28 extends transverse to the spinal rod 16. The second clamping member 26 is adjustable along the connecting rod 28 while the first clamping member 24 is connected to the spinal rod 14.

The second clamping member 26 has an outer surface 67 (FIGS. 2-5). The outer surface 67 comprises a top portion 68, a base portion 69, a posterior portion 70 (FIG. 3), a frontal portion 72 and first and second lateral portions 74, 76. The portions 68, 69, 70, 72, 74, and 76 meet at edges 78. The edges 78 are beveled. Accordingly, the second clamping member 26 does not have sharp edges.

The second clamping member 26 includes a spinal rod receiving channel 80 which receives the spinal rod 16. The second clamping member 26 is fastened to the spinal rod 16 by a second fastener or set screw 82 received in a bore 84. The bore 84 in the second clamping member 26 extends through the posterior portion 70 of the outer surface 67.

The set screw 82 has external threads which engage internal threads in the bore 84 of the second clamping member 26. The head 90 of the set screw 82 can accommodate a tool, such as a hex driver or an Allen wrench.

The spinal rod receiving channel 80 of the second clamping member 26 has a height which is at least as large as the diameter of the spinal rod 16. The spinal rod receiving channel 80 has a contour which matches the contour of the spinal rod 16. The set screw 82 of the second clamping member 26 protrudes slightly into the connecting rod opening 65 (FIG. 5).

The connecting rod opening 65 is axially transverse to the spinal rod receiving channel 80. The connecting rod opening 65 has a height which is at least as large as the second cross-section 64 of the connecting rod 28. The connecting rod opening 65 has a contour which matches the contour of the connecting rod 28. As the set screw 82 is tightened relative to the second clamping member 26, the set screw applies force to press the connecting rod 28 against the spinal rod 16 and the spinal rod 16 into position against the second clamping member.

When the apparatus 10 is to be implanted, a surgeon connects the two spinal rods 14, 16 to the vertebral column 18 with the fixation elements 22. The surgeon then connects two transverse connectors 12 to the spinal rods 14, 16 (FIG. 1). The two transverse connectors 12 are connected one at a time. The transverse connectors 12 are connected to the spinal rods 14, 16 by first bending the connecting rods 28 into a desired shape. Then, the first clamping member 24 is connected to the spinal rod 14 and the set screw 44 is loosely tightened. The second clamping member 26 is then placed on the connecting rod 28 by sliding the rounded end 66 of the connecting rod into the connecting rod opening 65 of the second clamping member 26. The set screw 82 is loosely tightened.

The second clamping member 26 is axially adjustable along the connecting rod 28. Once the surgeon determines the desired axial position of the second clamping member on the connecting rod 28, the spinal rod 16 is received in the spinal rod receiving channel 80 of the second clamping member 26. The surgeon then finishes tightening the set screws 44, 82 to securely connect the transverse connector 12 to the spinal rods 14, 16.

The connecting rod 28 extends transverse to and between the two spinal rods 14, 16. The connecting rod 28 is integral with the first clamping member 24 so that the second clamping member 26 can be slid onto the connecting rod without having to disconnect the first clamping member from the spinal rod 14. Accordingly, the transverse connector 12 is easily connected to the spinal rods 14, 16.

From the above description of the invention, those skilled in the art will perceive improvements, changes and modifications. Such improvements, changes and modifications within the skill of the art are intended to be covered by the appended claims.

Having described the invention, the following is claimed:

1. An apparatus for use in spinal corrective surgery comprising:
two spinal rods for at least partially extending along a vertebral column;
a plurality of fixation elements for fixing said spinal rods to the vertebral column; and
a transverse connector for connecting said spinal rods to each other, said transverse connector having:
a first clamping member with a spinal rod receiving channel for receiving one of said spinal rods;
a connecting rod integral with said first clamping member, said connecting rod extending transverse to said spinal rods, said connecting rod having a tapered portion extending from said first clamping member;
a first fastener for connecting said one of said spinal rods to said first clamping member;
a second clamping member including a spinal rod receiving channel for receiving another one of said spinal rods, said second clamping member including an opening for receiving said connecting rod; and
a second fastener for connecting said other one of said spinal rods and said connecting rod to said second clamping member.

2. The apparatus according to claim 1, wherein said second fastener applies force to said connecting rod to press said connecting rod against said one of said spinal rods and said one of said spinal rods against said second clamping member.

3. The apparatus according to claim 1, wherein said first and second clamping members have beveled edges.

4. The apparatus according to claim 1, wherein said tapered portion extends from said first clamping member opposite said spinal rod receiving channel.

5. The apparatus according to claim 1, wherein said tapered portion has a first cross-section adjacent said first clamping member and a second cross-section spaced from said first clamping member, said first cross-section being larger than said second cross-section.

6. The apparatus according to claim 1, wherein said connecting rod has a rounded end located opposite said tapered portion.

7. The apparatus according to claim 1, wherein said second clamping member is adjustable along said connecting rod while said first clamping member is connected to said one of said spinal rods.

8. The apparatus according to claim 1, wherein said transverse connecting rod is deformable.

9. A transverse connector for interconnecting spinal rods extending along a vertebral column, said transverse connector comprising:
a first clamping member including a spinal rod receiving channel for receiving one of the spinal rods;
a connecting rod integral with said first clamping member, said connecting rod extending transverse to the spinal rods, said connecting rod having a tapered portion extending from said first clamping member;
a first fastener for connecting the one of the spinal rods to the first clamping member;
a second clamping member including a spinal rod receiving channel for receiving another one of the spinal rods, said second clamping member including an opening for receiving said connecting rod; and
a second fastener for connecting the other one of the spinal rods and said connecting rod to said second clamping member.

10. The apparatus according to claim 9, wherein said second fastener applies force to said connecting rod to press said connecting rod against said one of said spinal rods and said one of said spinal rods against said clamping member.

11. The apparatus according to claim 9, wherein said first and second clamping members have beveled edge portions.

12. The apparatus according to claim 9, wherein said tapered portion extends from said first clamping member opposite said spinal rod receiving channel.

13. The apparatus according to claim 9, wherein said tapered portion has a first cross-section adjacent said first clamping member and a second cross-section spaced from said first clamping member, said first cross-section being larger than said second cross-section.

14. The apparatus according to claim 9, wherein said connecting rod has a circular cross-section and has a rounded end located opposite said tapered portion.

15. The apparatus according to claim 9, wherein said second clamping member is adjustable along said connecting rod while said first clamping member is connected to the one of the spinal rods.

16. The apparatus according to claim 9, wherein said connecting rod is deformable.
